(19) United States
(12) Patent Application Publication
(10) Pub. No.: US 2009/0281584 A1
(43) Pub. Date: Nov. 12, 2009

(54) IMPLANTABLE SENSOR ARRANGEMENT

(76) Inventors: Andreas Blomqvist, Spanga (SE); Anders Bjorling, Solna (SE); Göran Budgivars, Spanga (SE); Dominic Rivas, Sundbyberg (SE); Maria Torpo, Sundbyberg (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/519,416

(22) PCT Filed: Dec. 20, 2006
(86) PCT No.: PCT/SE06/01468

§ 371 Date: Jun. 16, 2009

(51) Int. Cl. A61N 1/362; A61B 5/04; A61N 1/39; H01R 43/00

(52) U.S. Cl. 607/4; 600/373; 29/592.1

(57) ABSTRACT

An implantable medical sensor arrangement has a sensor body configured for implantation in a subject, to which at least one sensor head is connected through at least one connective wire. The sensor head(s) and at least a portion of the connective wire(s) are tightly packed and enclosed by a protective sensor shell. This sensor shell is composed of a dissolvable material that will dissolve or can be triggered to dissolve following introduction of the sensor arrangement into a subject.

### BACKGROUND OF THE INVENTION

1. Field of the Invention

The present invention generally relates to sensor arrangements, and in particular to implantable sensor arrangements that can be easily introduced into a subject.

2. Description of the Prior Art

There is an ever increasing trend of employing implantable sensors in subjects for measuring different physiological parameters and other quantities of diagnostic and medical interest. For example, implantable medical devices, such as pacemakers, implantable cardioverters or implantable defibrillators, are typically connected, through leads, with different sensors for measuring electrical and blood-related parameters in subjects. Thus, there may be situations or particular subjects, in which multiple different sensor measurements would be advantageous during given periods of time. Today, this is traditionally solved by visiting a physician for conductance of a routine examination. However, this is a time- and cost-consuming solution. In addition, it might be useful or even necessary to derive sensor measurements over time to determine or detect certain trends in the monitored parameters. There is therefore a need of implanting multiple sensors in a subject.

However, introducing multiple sensors is not without risks or problems. Firstly, there is the problem of manually handling multiple flexible sensors and being able to insert them at a correct measuring location, typical a blood vessel. The sensors and sensor connections cannot be too stiff, but must generally be flexible to cope with and adjust to movements of the vessel or other implantation site. Secondly, implanting many sensors in a blood vessel may cause occlusion of the vessel and redirection of the blood. Aside from causing changes to the blood flow, no correct readings from the sensor would be possible due to the lack of a blood flow. Thirdly, sensors and sensor heads are generally delicate structures that can be damaged during implantation, especially when multiple sensors are to be implanted at the same site in a subject.

### SUMMARY OF THE INVENTION

The present invention overcomes these and other drawbacks of the prior art arrangements.

It is a general object of the present invention to provide an improved sensor arrangement.

It is another object of the invention to provide a sensor arrangement that can be easily introduced into a subject.

Yet another object of the invention is to provide a sensor arrangement that will protect sensitive sensors during implantation in a subject.

It is a particular object of the invention to provide a sensor arrangement having multiple different sensors and still being easily handled.

Briefly, the present invention involves a sensor arrangement having a sensor body. At least one sensor head, preferably multiple different sensor heads, are connected to the sensor body with at least one connective wire. The sensor head(s) and at least a portion of the connective wire(s) are tightly packed into a small volume that is enclosed by a protective sensor shell. This sensor shell is of a dissolvable material that will (spontaneously) dissolve or can be triggered to start dissolving following introduction of the protective sensor shell and the enclosed sensor head(s) into a subject body.

The sensor shell will protect the often very sensitive sensor heads from mechanical damage during the implantation procedure. In addition, the shell will dramatically facilitate handling of the otherwise unruly and highly flexible wire-sensor entity. This therefore allows for introduction of several sensors into tight measuring sites, such as blood vessel. As the protective shell will dissolve following the implantation, the risk of occlusion is next to minimized and the sensor heads can flow freely in the blood vessel.

Other advantages offered by the present invention will be appreciated upon reading of the below description of the embodiments of the invention.

### SHORT DESCRIPTION OF THE DRAWINGS

The invention together with further objects and advantages thereof, may best be understood by making reference to the following description taken together with the accompanying drawings.

FIG. 1 is an illustration of an end portion of a sensor arrangement according to an embodiment of the present invention.

FIG. 2 is an illustration of the sensor arrangement of FIG. 1 without the dissolvable protective shell according to an embodiment of the present invention.

FIG. 3 is an illustration of the sensor arrangement of FIG. 1 without the dissolvable protective shell according to another embodiment of the present invention.

FIG. 4 is an illustration of an end portion of a sensor arrangement according to another embodiment of the present invention.

FIG. 5 is an illustration of the sensor arrangement of FIG. 4 without the dissolvable protective shell according to an embodiment of the present invention.

FIG. 6 is an illustration of an end portion of a sensor arrangement according to a further embodiment of the present invention.

FIG. 7 is a schematic block diagram of an implantable medical device equipped with a sensor arrangement according to the present invention.

FIG. 8 is an illustration of a human subject having an implantable medical device equipped with a sensor arrangement according to the present invention.

FIG. 9 is a flow diagram of a method of manufacturing a sensor arrangement according to the present invention.

FIG. 10 illustrates a method of introducing a sensor arrangement according to the present invention into a subject body.

### DESCRIPTION OF THE PREFERRED EMBODIMENTS

Throughout the drawings, the same reference characters will be used for corresponding or similar elements.

The present invention relates to a sensor arrangement that allows easy introduction into a subject, such as animal or human subject, where sensor measurements and parameter monitoring, preferably multiple parallel such measurements and monitorings are desired.

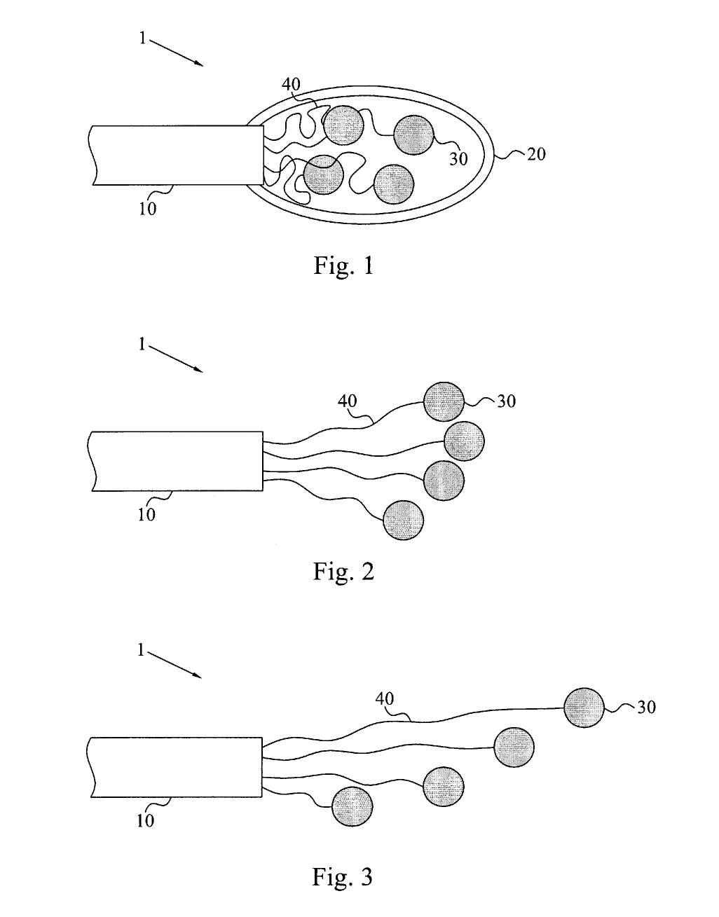

FIG. 1 illustrates an end portion of a sensor arrangement 1 according to an embodiment of the present invention. The sensor arrangement 1 has a sensor body 10 that could be, for example, a lead, electrode or probe connected to an implantable medical device. Alternatively, the sensor body 10 constitutes an independent unit containing battery or other power source required for powering at least one sensor head 30 of the arrangement 1. In an implementation, the sensor body 10 also comprises a transmitter for transmitting measurement data collected by the at least one sensor head 30. Alternatively, or in addition, the sensor body 10 can include a data memory for storing measurement results from the sensor head(s) 30, thereby allowing extraction of the measurement data at a later explantation occasion.

According to the present invention, at least one, preferably multiple, i.e. at least two, sensor heads 30 are connected to the sensor body 10 with at least one connective wire 40. These sensor heads 30 constitute the actual measuring elements of the sensor arrangement 1 for determining, measuring and/or monitoring different physiological parameters and other quantities of diagnostic and medical interest. In FIG. 1, each sensor head 30 has a dedicated connective wire 40 connecting the head 30 with the sensor body 10. The measurement data registered in the heads 30 are forwarded on the wires 40 into the sensor body 10 for storage therein or transmission to an external (non-implanted) receiver.

According to the present invention, the sensor heads 30 and at least a portion of the connective wires 40 are enclosed by a protective sensor shell 20. This sensor shell or enclosure 20 allows simple management and introduction of the multiple sensors 30 and wires 40 into a given implantation site since these otherwise unruly sensors 30 and wires 40 can be handled as a single unit. The sensor shell 20 also protects the sensitive sensor heads 30 during transportation, storage and the actual implantation procedure. The sensor shell 20 is preferably attached to the sensor body 10 and therefore encloses all the sensor heads 30 and the connective wires 40.

The sensor shell 20 of the present invention is further made of a dissolvable material. According to the present invention “dissolvable” material encompasses materials constituted to spontaneously or be caused to dissolve, degrade or resorb following implantation in a subject. The dissolvable material could start to dissolve upon contact with a selected agent that is preferably a body fluid, such as blood or lymph. In such a case, the protective shell 20 will tightly keep the sensor head 30 and wires 40 closely packed during implantation. However, as the shell 20 comes into contact with a body fluid, it starts dissolving. The dissolving rate of the protective shell 20 is affected both by the particular material of the shell 20 and the thickness of the shell 20. Given a dissolvable material, the thickness of the shell 20 is preferably selected to allow time for (e.g. intravascular or intralymphatic) insertion of the sensor heads 30 and connective wires 40 into the implantation site (e.g. blood vessel or lymphatic vessel).

Thus, the time following the start of dissolving the sensor shell 20 until the shell 20 is fully dissolved can be pre-determined by selecting the particular thickness of the protective shell 20. In most typical implementation this time would correspond to one or few minutes up to one or few hours, possibly even up to one or few days or even longer.

Typical materials having this dissolvable property include sugar derivates, such as mannitol, dextrose, sorbose, sucrose and glucosamine. Alternatively, salts, such as sodium chloride, potassium chloride or sodium carbonate could be employed. Further examples of dissolvable material according to the invention include polymer materials such as proteins/amino acid polymers, polyhydroxycarboxy acids, carbohydrate polymers and polyvinylpyrrolidone. Suitable proteins/amino acid polymers include gelatin, collagen, polyserine, polythreonine or polyphenylalanine. Polylactides or polyglycolides can be used as preferred polyhydroxycarboxyl acids. A carbohydrate polymer can be selected from dextran, starch, hyaluronic acid or cellulose.

These dissolvable materials will start dissolving spontaneously upon contact with a selected agent or body fluid. However, the present invention could alternatively use dissolvable materials where the dissolving procedure is actively triggered. In such a case, the initiation of the dissolve can be controlled to only start once the sensor arrangement has been arranged at the desired measurement site. A typical example of such a material is a material that starts to dissolve upon application of an energy pulse. For example, following implantation, an ultrasound pulse could be applied onto the surface (skin) of the subject directly above the sensor shell location in the subject. The mechanical pulse from the ultrasound source can initiate or speed up the dissolving of the sensor shell 20. An example of suitable sensor shell material in this case includes one of the above sugar derivates. Alternatively, a current pulse could be applied to the sensor body 10 or using a separate lead brought in contact or close vicinity of the sensor shell 20.

In a further embodiment of the invention, an agent that triggers or speeds up the dissolving of the sensor shell 20 could be provided through the sensor body 10. The sensor body 10 then preferably has an internal channel or an external structure defining such a channel, through which the agent can be transported to the shell 20. In the former case, the agent will be injected into the internal space or volume defined by the shell 20 and includes the sensor heads 30 and the wiring 40. In the latter case, the agent could alternatively be applied to an outside surface of the sensor shell 20. Examples of agents that can be provided in this manner include agents that affect the local environment in connection with the sensor shell 20, such as pH modifying agents or salt concentration modifying agents. This agent can have the effect of modifying, i.e. increasing, the reaction rate of the shell dissolving. It is anticipated by the invention that the agent should be biocompatible and not toxic at the amounts employed for triggering or enhancing the dissolving of the sensor shell 20.

The sensor shell 20 can include different kinds of dissolvable material as described above, for example having an inner sensor shell of a first dissolvable material and an outer shell of a second dissolvable material. In this manner different advantageous properties of the dissolvable materials of the invention can be fully exploited to cope with the demands of the actual subject, sensor types, implantation site, etc.

In addition to being dissolvable, the sensor shell material is of course non-toxic and bio-compatible. If the material is degraded into reaction products following implantation, these products are preferably also non-toxic and bio-compatible to not cause any deleterious reactions or at most mild temporary reactions in the subject body.

The sensor shell 20 preferably has a flexible, deformable structure to allow easy introduction of the shell 20 at the desired implantation site without damaging blood vessels, lymphatic vessels or other organs and tissues at the site or encountered during the insertion of the sensor arrangement 1.

By tightly packing the sensor heads 30 and the connective wires 40 inside the protective sensor shell 20, the risk of occlusion of vessel during the actual implantation procedure is minimized as the total size of the shell 20 can be kept small.

When the sensor arrangement 1 is introduced at the measurement site in a subject, the dissolvable material is starting to or is actively caused to start dissolving for releasing the sensor heads 30 and connective wires 40, as illustrated in FIG. 2. When the protective sensor shell is fully dissolved as in FIG. 2, the separate sensor heads 30 are released and are now allowed to adapt to and flow freely in the measurement/implantation site, such as a blood stream. At this point, the occlusion is effectively prevented as the sensors will adapt to and not block the blood stream.

In FIG. 2, each sensor head 30 is connected to the sensor body 10 with a respective connective wire 40. As is illustrated in FIG. 2, the connective wires 40 are of a generally equal length. However, in order to prevent the wires 40 and sensors 30 from becoming too tangled and further to prevent the sensor heads 30 from negatively interact or collide with each other and become damaged, at least two of the connective wires 40, preferably all of the wires 40, have different wire lengths as illustrated in FIG. 3. In FIG. 3, the different sensor heads 30 end up at different distances from the sensor body end. No or only marginal (possibly negative) interaction between the sensors 30 will arise with this configuration. In addition, the risk of colliding sensor heads 30 as the subject moves or in response to the pumping of blood through a vessel is minimized by having different wire lengths.

The connective wires 40 attaching the sensor heads 30 to the sensor body 10 can be traditional connective (electrical) wires 40 employable in transplantation implementations. In a particular embodiment of the invention, at least one of the wires 40, preferably all connective wires 40, are elastic or springy. This allows for, following the dissolving of the protective sensor shell, efficient positioning of the sensor heads 30 at the implantation site, in particular if implanted in a body vessel. After dissolving the shell, the elastic property of the wires 40 will cause the sensor heads 30 to be pushed away from the sensor body 10 and into correct measuring positions distanced away from the body 10. In addition, depending on the particular elasticity of the wires 40, the sensor heads 30 can exert a force against the inside of the protective sensor shell, thereby speeding up the dissolving and breaking of the sensor shell.

The elasticity of the wires 40 helps in avoiding the wires 40 from getting entangled and thereby reduces the risk of having sensor heads 30 that mechanically interfere each other or interfere each other from a measurement technical point of view. The elasticity can also have positive effects in the fixation of the sensor arrangement 1 at the measuring/implantation site.

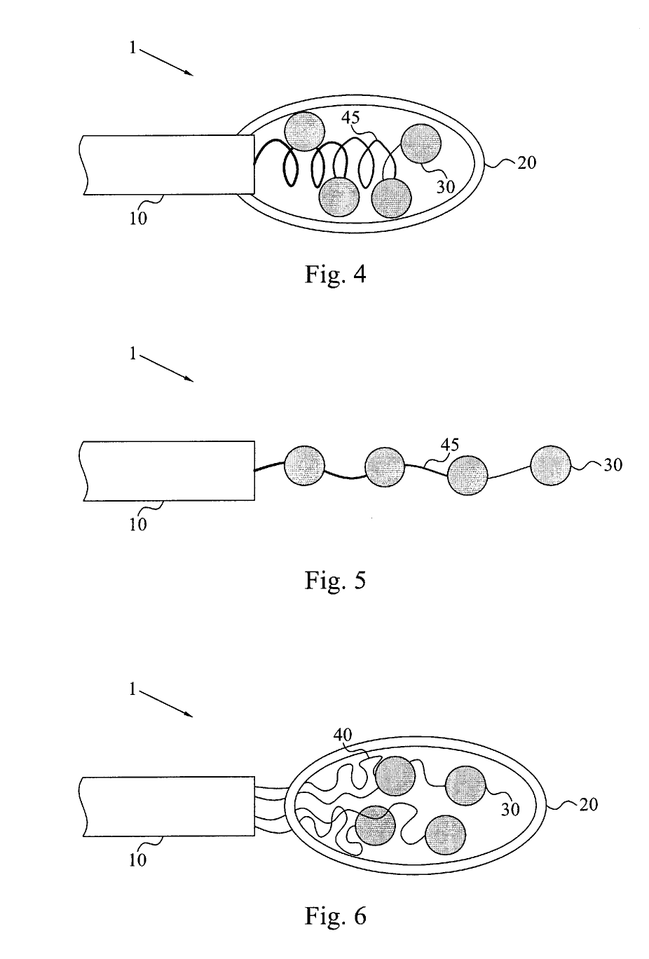

FIG. 4 is an illustration of another embodiment of the sensor arrangement 1 of the present invention. This sensor arrangement 1 utilizes a coiled multi-wire 45 onto which at least one, preferably multiple, sensor heads 30 are arranged. The multi-wire 45 with the sensors 30 is tightly packed into a protective sensor shell 20 of a dissolvable material. FIG. 5 is a corresponding illustration of the sensor arrangement 1 following dissolving the sensor shell. The sensor heads 30 are then positioned on the multi-wire 45 like beads on a string. In a preferred implementation, the multi-wire 45 comprises multiple wires of different lengths. Each sensor head 30 is then connected to a respective wire of the multi-wire 45. As a result, the sensor heads 30 will be positioned at different distances along the multi-wire 45 from the sensor body 10. This embodiment has the advantage of minimizing the risk of the wires becoming tangled or attaching to each other. A disadvantage in some instances could be that it behaves less flexibly as compared to having multiple wires.

The protective sensor shell 20 of the present invention does not necessarily have to be attached or anchored to the sensor body 10. FIG. 6 illustrates another possible embodiment, in which the sensor shell 20 encloses the sensor head(s) 30 and only a portion of the connective wire(s) 40. The end portions of the wires 40 closest to the connection at the sensor body 10 are therefore free and not enclosed by the shell 20. This embodiment may be somewhat more cumbersome to use when inserting the sensor arrangement 1 into a desired target site in a subject as the shell 20 is separate from and movable relative the body 10. However, for certain target sites this embodiment may still be effectively employed without major problems.

The sensor arrangement of the present invention can be used in connection with a vast multitude of different implantable sensors. Today, several such sensor solutions are available from different manufactures. Furthermore, there is a general trend towards reducing the overall sizes of the implantable sensors, which is an advantage in connection with the present invention. The sensor arrangement of the invention has the further advantage that it can be connected to or forming part of an implantable medical device (IMD). This means that cumbersome elements, such as sensor battery, transmitter equipment and/or data storage, could be implemented in the IMD body instead of in the sensors. This further allows for very small overall sensor sizes.

Non-limiting sensor examples that can be used in connection with the present invention are sensors that are adapted for measuring or monitoring at least one of the following quantities or physical parameters: pressure; temperature; current; voltage; impedance; blood glucose; oxygen; different metabolites; specific drugs or medicaments; electrolytes, such as sodium, potassium, carbon dioxide, chloride; creatinine; blood urea nitrogen (BUN); high-density lipoprotein; low-density lipoprotein; bilirubin, etc. Other sensor examples include activity sensors; optical sensors; microphones; vibration sensors; acceleration sensors; stretch sensor; etc.

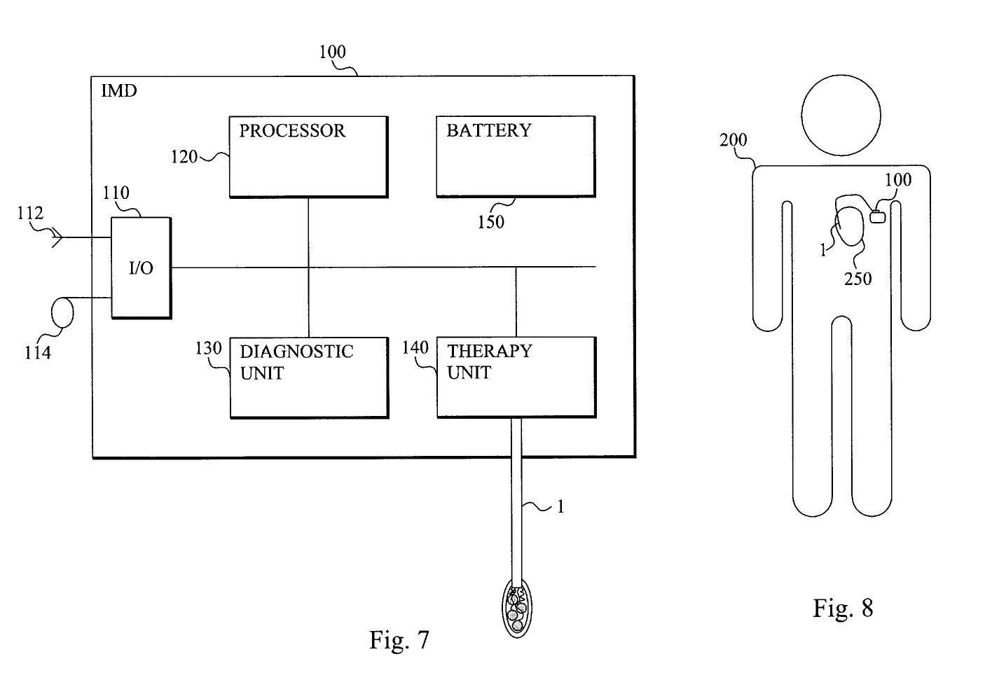

FIG. 7 is a schematic block diagram of an implantable medical device 100 comprising or being connected to a sensor arrangement 1 according to the present invention. The IMD 100 may be a pacemaker, implantable cardioverter or implantable defibrillator for applying heart therapy in the form of heart stimulation in a patient in need thereof. The IMD 100 need not necessarily be employed for heart therapy but can be used for stimulating other body tissues, e.g. be a neurological stimulator.

The IMD 100 generally includes an input and output (I/O) unit 110 (transmitter/receiver chain) for conducting wireless communication with an external unit, e.g. a programmer. This I/O unit 110 includes functionalities for processing incoming and outgoing data messages, optionally including modulator/demodulator and coder/decoder functionality. The I/O unit 110 is further preferably connected to an antenna arrangement 112 used for transmitting and receiving radio packets to and from the external unit, respectively. However, the I/O unit 110 could also or alternatively use other forms of wireless techniques than radio frequency transmissions when communicating with the external device. The I/O unit 110 could for example use an inductive antenna 114 for external wireless communication.

In a preferred embodiment of the invention, the IMD 100 also has a diagnostic unit 130 for processing physiological data collected by a sensor arrangement 1 according to the present invention. The sensor arrangement 1 could therefore be a probe directly connected to the diagnostic unit 130. However, it is also or alternatively possible to implement the sensor arrangement 1 of the invention connected to or forming a part of a lead used for delivering therapy. In either case, the collected and measured physiological parameter data is then forwarded to the processor 120 for data processing. The processor 120 will determine, based on the collected physiological data, whether there is a need for tissue stimulation. For example, collected data of the operation of a patient's heart may indicate heart arrhythmia and a need for heart stimulation. In such a case, the processor 120 generates a stimulation signal that is forwarded to a therapy unit 140 connected to the lead or to multiple leads.

The IMD 100 is also typically equipped with a battery 150 or other power source for providing the power necessary for driving the I/O unit 110, processor 120, diagnostic unit 130, therapy unit 140 and sensors of the sensor arrangements 1.

The sensor arrangement 1 of the invention may constitute a separate device that is connectable to the IMD 100, the diagnostic unit 130 or the therapy unit 140. Alternatively, the arrangement 1 constitutes an internal part of the IMD 100 and cannot be reversibly be detached therefrom. In such a case, the relatively larger size of the IMD 100 can be used while reducing the size of the sensor arrangement 1. For example, the powering, data processing and data storing functionality used in connection with the sensor arrangement 1 could be physically implemented in the IMD body 100.

The units 110, 120, 130 and 140 of the IMD 100 can be provided as hardware, software or a combination of hardware and software.

FIG. 8 schematically illustrates an IMD 100 with a sensor arrangement 1 of the invention implanted in a patient or subject 200 in need thereof. In the figure, the IMD 100 is illustrated as a device that monitors and/or provides therapy to the heart 250 of the patient 200, such as a pacemaker, defibrillator or cardioverter. As a consequence, the sensor arrangement 1 could be used for measuring or monitoring different parameters representative of the operation and condition of the patient heart 250 or relevant for the operation of the IMD 100.

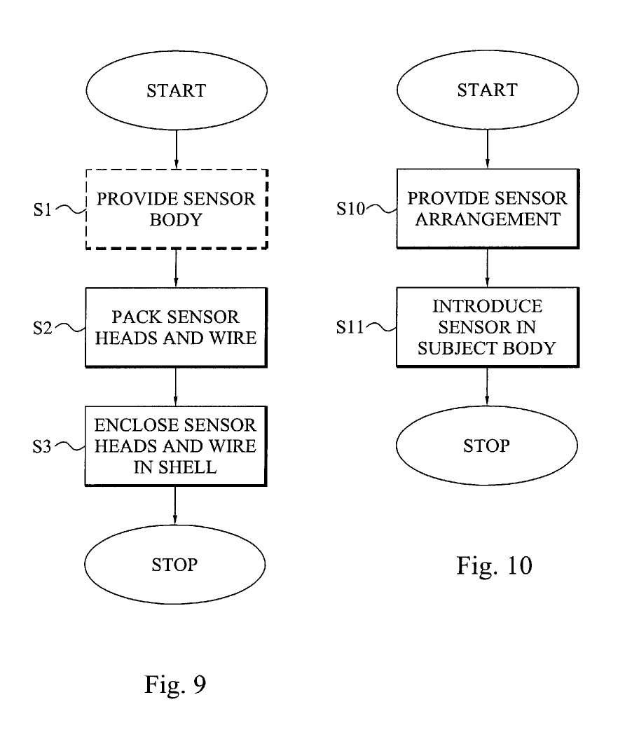

FIG. 9 is a flow diagram illustrating a method of manufacturing a sensor arrangement according to the present invention. The method generally starts in the optional step S1, where a sensor body having at least one connected sensor head and where the connection is realized by at least one connective wire. In a preferred embodiment, multiple sensor heads are connected to the sensor body using multiple connected or separate connective wires. In a next step S2, the sensor heads and the wires are packed close together into a limited volume. This limited volume is then enclosed by a protective sensor shell of a dissolvable material to form the sensor arrangement of the present invention. The method then ends.

In a first embodiment of this enclosing step S3, the whole lengths of the connective wires are enclosed together with the sensors heads in the protective shell. In a second embodiment, only a portion of the connective wires are enclosed with the heads. The protective shell could be formed connected and attached to the sensor body or as a separate entity.

Different techniques could be employed for forming the protective sensor shell, including dip coating process or formed separately by casting or injection molding for later attachment to the sensor body using an adhesive.

In the former case, the dissolvable material of protective sensor shell is in a fluid state above a given melting temperature and solid below this temperature. This melting temperature is preferably higher than general room temperature (i.e. higher than about 20-25° C.) but not so high as to damage the sensor heads when they come into contact with the fluid material.

The enclosing step then involves heating the dissolvable material to a temperature slightly above its melting point. The tightly packed sensor heads and connective wires (possible also an end part of the sensor body) are dipped into the solution of the material maintained at the elevated temperature. The sensor body with the sensor heads and connective wires are then removed, along with an initial portion of the sensor shell adhering thereto, from the solution and permitted to cool a sufficient time for the attached dissolvable material to at least partly solidify. This procedure is repeated to add more dissolvable material to form the protective sensor shell of the invention. The number of dipping occasions determines the thickness of the protective sensor shell and thereby the dissolving time of the shell when introduced into a subject.

For a more improved control over the size and shape of the protective sensor shell, the shell can be formed by casting or injection molding of the dissolvable material. The resulting shell is then fixed to the sensor body by a suitable adhesive. This adhesive can be molten dissolvable material itself or some other adhesive that is compatible with the sensor body material and the dissolvable material(s) of the sensor shell. This embodiment has the advantage of affording maximum control of the sensor shell size and shape and in particular sensor shell thickness that affects the total dissolving time of the shell. A further advantage is that less dissolvable material are generally required as compared to the dip coating process.

FIG. 10 is a flow diagram illustrating a method of introducing a sensor arrangement of the present invention into a subject, such as an animal or human subject, preferably a human subject, in which different quantities or parameters should be measured or monitored. The method starts in step S10, where a sensor arrangement of the invention is provided. Thus, this sensor arrangement comprises a sensor body to which at least one, preferably multiple different sensor heads are attached through at least one connective wire. The sensor heads and wire(s) are further enclosed in a protective sensor shell of a dissolvable material that starts to dissolve or can be triggered to start dissolving following introduction of the sensor shell in the subject.

In a next step S11, at least a portion of the sensor arrangement is introduced into a selected position or site in the body of the subject, where the sensors should measure the physiological parameters or quantities. In this introducing step, only the end portion of the arrangement containing the sensor shell enclosing the sensor heads and connective wires could be inserted into the body. However, in an alternative embodiment the whole sensor arrangement is implanted in the subject body.

The sensor arrangement of the present invention can be implanted or inserted at different target sites of a subject, depending on the particular parameters to be measured. Typical exemplary sites include, but are not limited to, blood vessels; lymphatic vessels; in or in connection with different organs and tissues, such as heart, kidney, liver and spleen.

Although modifications and changes may be suggested by those skilled in the art, it is the intention of the inventors to embody within the patent warranted heron all changes and modifications as reasonably and properly come within the scope of their contribution to the art.

1. A sensor arrangement comprising:
a sensor body;
at least one sensor head connected to said sensor body with at least one connective wire; and
a protective sensor shell enclosing said at least one sensor head and at least a portion of said at least one connective wire, wherein said protective sensor shell is of a dissolvable material.

2. The arrangement according to claim 1, further comprising multiple sensor heads connected to said sensor body with said at least one connective wire.

3. The arrangement according to claim 2, wherein said at least one connective wire is a coiled multi-wire comprising one individual wire per sensor head.

4. The arrangement according to claim 2, further comprising multiple connective wires and wherein each sensor head of said multiple sensor heads is connected to an individual wire of said multiple connective wires.

5. The arrangement according to claim 4, wherein said multiple connective wires have different wire lengths.

6. The arrangement according to claim 1, wherein said dissolvable material is a material that starts to dissolve upon contact with a selected agent.

7. The arrangement according to claim 6, wherein said selected agent is body fluid.

8. The arrangement according to claim 1, wherein said dissolvable material is a material that starts to dissolve upon application of an energy pulse.

9. (canceled)

10. The arrangement according to claim 1, wherein said protective sensor shell encloses said at least one sensor head and said at least one connective wire.

11. The arrangement according to claim 1, wherein said protective sensor shell is connected to said sensor body.

12. The arrangement according to claim 1, wherein said protective sensor shell has a flexible, deformable structure.

13. The arrangement according to claim 1, wherein said sensor body is configured as a lead of an implantable medical device.

14. The arrangement according to claim 1, wherein said at least one connective wire is at least one elastic connective wire.

15. (canceled)

16. (canceled)

17. A method of manufacturing a sensor arrangement comprising the steps of:
providing a sensor body having at least one sensor head connected to said sensor body with at least one connective wire; and
enclosing said at least one sensor head and at least a portion of said at least one connective wire with a protective sensor shell of a dissolvable material.

18. The method according to claim 17, wherein said enclosing step comprises enclosing said at least one sensor head and said at least one connective wire with said protective sensor shell.

19. The method according to claim 17, wherein said enclosing step comprises enclosing said at least one sensor head and said at least a portion of said at least one connective wire with said protective sensor shell by connecting at least a portion of said protective sensor shell to said sensor body.

20. A method of introducing a sensor arrangement into a subject comprising the steps of:
providing a sensor arrangement having a sensor body carrying at least one sensor head connected to the sensor body with at least one connective wire, with said at least one sensor head and at least a portion of said at least one connective wire being enclosed with a protective sensor shell of a dissolvable material; and
introducing at least a portion said sensor arrangement into a selected position of a body of said subject.

21. The method according to claim 20, wherein said introducing step comprises introducing a protective sensor shell enclosing at least one sensor head into a blood vessel or lymphatic vessel of said subject.

22. The arrangement according to claim 1 wherein said dissolvable material is a material selected from the group consisting of sugar derivatives, salts, and polymer materials.

23. The arrangement according to claim 1 wherein said dissolvable material is a sugar derivative selected from the group consisting of mannitol, dextrose, sorbose, sucrose and glucosamine.

24. The arrangement according to claim 1 wherein said dissolvable material is a salt selected from the group consisting of sodium chloride, potassium chloride and sodium carbonate.

25. The arrangement according to claim 1 wherein said dissolvable material is a polymer material selected from the group consisting of amino acid polymers, polyhydroxycarboxyl acids, carbohydrate polymers, and polyvinylpyrrolidone.

26. The arrangement according to claim 1 wherein said dissolvable material is an amino acid polymer selected from the group consisting of gelatin, collagen, polyserine, polythreonine and polyphenylalanine.

27. The arrangement according to claim 1 wherein said dissolvable material is a polyhydroxycarboxyl acid selected from the group consisting of polylactides and polyglycolides.

28. The arrangement according to claim 1 wherein said dissolvable material is a carbohydrate polymer selected from the group consisting of dextran, starch, hyaluronic acid, and cellulose.

29. An implantable medical device comprising:
a housing configured for in vivo implantation in a subject;
a therapy administration device at least partially contained in said housing and configured to administer therapy in vivo to the subject;
a sensor arrangement configured for in vivo interaction with the subject, comprising a sensor body, at least one sensor head connected to said sensor body with at least one connective wire, and a protective sensor shell enclosing said at least one sensor head and at least a portion of said at least one connective wire, said protective sensor shell being comprised of a dissolvable material; and
a control unit connected to said therapy administration device and to said sensor arrangement, that controls administration of said therapy by said therapy administration device dependent on a sensor signal generated by said at least one sensor head and supplied to said control unit via said at least one connective wire.

30. A medical device as claimed in claim 29 wherein said therapy administration unit is selected from the group consisting of pacemakers, cardioverters, and defibrillators.
